(19) United States
(12) Patent Application Publication
(10) Pub. No.: US 2026/0294463 A1
(43) Pub. Date: Oct. 1, 2026

(54) TECHNOLOGIES FOR ROTATING ELECTRICAL CONTACTS ON SURGICAL DEVICES TO DELIVER ENERGY TO TISSUE

(71) Applicant: Cilag GmbH International, Zug (CH)

(72) Inventors: Tyler N. Brehm, Cincinnati, OH (US); Jacob Gee, Cincinnati, OH (US); Christopher Bull, Cincinnati, OH (US); Andrew Meyers, Cincinnati, OH (US); Cameron D. McLain, Cincinnati, OH (US); Barry Worrell, Cincinnati, OH (US); Thor Storm, Cincinnati, OH (US); Ryan Reese, Cincinnati, OH (US); Ellen Burkart, Cincinnati, OH (US); Matthew Bruchas, Cincinnati, OH (US); Cole Grabowski, Cincinnati, OH (US)

(21) Appl. No.: 19/410,615

(22) Filed: Dec. 5, 2025

Related U.S. Application Data
(60) Provisional application No. 63/780,379, filed on Mar. 30, 2025.

(51) Int. Cl. A61B 17/32; A61B 17/00

(52) U.S. Cl. CPC A61B 17/320092; A61B 2017/00477; A61B 2017/320071; A61B 2017/320093; A61B 2017/320094

(57) ABSTRACT

A surgical instrument for performing an energy-based surgical procedure includes an elongated shaft assembly, a trigger assembly, and a rotatable electrical connector. The elongated shaft assembly includes an outer tubular sheath and an end effector having a jaw assembly movable between an open state and a closed state. The trigger assembly is operable to move the jaw assembly of the end effector between the open state and the closed state and to apply bipolar energy to tissue captured within the jaw assembly. The rotatable electrical connector is configured to establish an electrical connection between the outer tubular sheath and an energy source of the bipolar energy. Additionally, the rotatable electrical connector is configured to facilitate rotation of the outer tubular sheath while maintaining the electrical connection between the outer tubular sheath and the energy source.

This application claims the benefit of and priority to U.S. patent application Ser. No. 63/780,379, entitled “TECHNOLOGIES FOR ROTATING ELECTRICAL CONTACTS ON SURGICAL DEVICES TO DELIVER ENERGY TO TISSUE,” WHICH WAS FILED on Mar. 30, 2025, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient's tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient's tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities, which increases the surgical options provided by the surgical instrument to the surgeon.

Some surgical instruments may include one or more rotating or otherwise movable parts. In such surgical instruments, an electrical connection may be required to be maintained across or through such rotating movable part. However, a rotating or movable electrical connections can be difficult to design to achieve a robust connection and reliable assembly. For example, under certain circumstances, typical rotating or movable electrical connections or contacts may be prone to failure, which can result in open circuits and failure of energy delivery to the tissue.

### SUMMARY

According to an aspect of the present disclosure, a surgical instrument for performing an energy-based surgical procedure includes an elongated shaft assembly, a trigger assembly, and a rotatable electrical connector. The elongated shaft assembly may include an outer tubular sheath and an end effector having a jaw assembly movable between an open state and a closed state. The jaw assembly may include an ultrasonic blade. The trigger assembly may be operable to move the jaw assembly of the end effector between the open state and the closed state and to apply bipolar energy to tissue captured within the jaw assembly. The rotatable electrical connector may be configured to establish an electrical connection between the outer tubular sheath and an energy source of the bipolar energy. The rotatable electrical connector may be configured to facilitate rotation of the outer tubular sheath while maintaining the electrical connection between the outer tubular sheath and the energy source.

In some embodiments, the rotatable electrical connector may be embodied as an elongated electrically conductive body having an eyelet connector at a first end and a tab connector at a second end opposite the first end. The eyelet connector may be configured to receive a male jack connector extending from a proximal end of a transducer of the surgical instrument. The eyelet connector may be configured to rotate about the male jack connector. In such embodiments, the rotatable electrical connector may further include an electrical interconnect having a first end electrically connected to the outer tubular sheath of the elongated shaft assembly and a second end, opposite the first end, electrically connected to a connector configured to mate with the tab connector of the elongated electrically conductive body. The elongated electrically conductive body and the electrical interconnect may cooperate to establish and electrical connection between the male jack connector of the transducer and the outer tubular sheath.

Additionally, in some embodiments, the rotatable electrical connector may be embodied as a carriage and a set of electrical pin contacts extending through carriage. In such embodiments, the carriage may be coupled to a housing of the surgical instrument and each electrical pin contact may include an electrically conductive pinhead in contact with the outer tubular sheath.

In some embodiments, the rotatable electrical connector may include an electrically conductive connector collar configured to couple with an electrically non-conductive rotation collar coupled to the outer tubular sheath, a carriage configured to couple to the electrically conductive connector collar, and a set of electrical pin contacts extending through carriage. The electrically non-conductive rotation collar may include a plurality of apertures. Additionally, the electrically conductive connector collar may include a plurality of tabs that extend axially inwardly. Each tab of the plurality of tabs may extend into a corresponding aperture of the plurality of apertures such that each tab is in contact with the outer tubular sheath. Furthermore, each electrical pin contact may include an electrically conductive pinhead in contact with the electrically conductive connector collar. In such embodiments, the rotatable electrical connector may further include a wire connector frame electrically coupled to each electrical pin contact and including a connector configured for electrical connection to an interconnect.

Additionally, in some embodiments, the rotatable electrical connector may be embodied as a cylindrical pin collar coupled to and encircling the outer tubular sheath and a set of electrical pin contacts extending through the cylindrical pin collar. Each electrical pin contact may include an electrically conductive pinhead in contact with the outer tubular sheath. In such embodiments, the electrically conductive pinhead of each electrical pin contact may be rounded or may have a concave surface that has a curvature that matches a curvature of an outside surface of the outer tubular sheath.

In some embodiments, the rotatable electrical connector may be embodied as an electrically conductive inner bearing ring attached to the outer tubular sheath, an electrically conductive outer bearing ring encircling the inner bearing ring, and a set of electrically conductive ball bearings positioned between the electrically conductive inner bearing ring and the electrically conductive outer bearing ring. In such embodiments, the rotatable electrical connector may further include an electrical interconnect electrically connected to the electrically conductive outer bearing ring. Additionally, each of the electrically conductive inner bearing ring and the electrically conductive outer bearing ring may be embodied as electrically conductive cylindrical bushings configured to facilitate axial movement of the outer tubular sheath through the rotatable electrical connector.

Additionally, in some embodiments, the rotatable electrical connector may include an electrically conductive spring clamp having a first arm extending around a first section of the outer tubular sheath and a second arm, opposite the first arm, extending around a second section of the outer tubular sheath opposite the first section. In some embodiments, each arm of the electrically conductive spring clamp may include an arm branch that extends axially inwardly and is configured to be deflected axially outwardly to facilitate connection of the electrically conductive spring clamp to the outer tubular sheath. Additionally or alternatively, each arm of the electrically conductive spring clamp may curve away from the other arm. Additionally, in some embodiments, each arm of the electrically conductive spring clamp may include a first arm branch and a second arm branch spaced from the first arm branch.

In some embodiments, the rotatable electrical connector may be embodied as an electrically conductive clip configured to attach to the outer tubular sheath. The electrically conductive clip may include a first arm extending around a first section of the outer tubular sheath and a second arm, opposite the first arm, extending around a second section of the outer tubular sheath opposite the first section. Each arm may include at least one detent that extends axially inwardly and is configured to contact the outer tubular sheath when the electrically conductive clip is attached to the outer tubular sheath. In such embodiments, the electrically conductive clip may further include a locking bracket configured to attach to each of the first arm and the second arm to lock the electrically conductive clip to the outer tubular sheath.

According to another aspect of the present disclosure, a method for monitoring operation of a surgical instrument may include determining, by a controller of the surgical instrument, whether (i) a jaw assembly of the surgical instrument is in a closed state and (ii) a bipolar energy has not been applied by the surgical instrument. The method may also include determining, by the controller, an impedance of tissue captured within the jaw assembly and comparing, by the controller, the determined impedance to a reference threshold impedance. The method may further include instructing, by the controller, a user of the surgical instrument to reduce bending of the surgical instrument in response to a determination that the determined impedance of the tissue is greater than the reference threshold impedance. In some embodiments, the reference threshold impedance may be about 20 ohms.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a block diagram of another embodiment of the control circuit of the surgical instrument of FIG. 2 including a rotatable electrical connector electrically connected to the outer sheath of the surgical instrument;

FIG. 9 is a perspective view of the surgical instrument of FIG. 8 showing internal components of the surgical instrument and including an embodiment of the rotatable electrical connector;

FIG. 10 is a perspective view of a female jack connector of the surgical instrument of FIG. 9 having a set of electrical contacts extending therefrom;

FIG. 11 is a side elevation view of a proximal end a transducer of the surgical instrument of FIG. 9 showing a male jack connector configured to be received in the female jack connector of FIG. 10;

FIG. 12 is a perspective view of an embodiment of the rotatable electrical connector of FIG. 8;

FIG. 13 is a side elevation view of the transducer of FIG. 11 including the rotatable electrical connector of FIG. 12 electrically coupled between the mail jack connector of FIG. 11 and the outer sheath of the surgical instrument of FIG. 8 via a movable electrical interconnect;

FIG. 14 is another side elevation view of the transducer of FIG. 12 including the rotatable electrical connector of FIG. 13;

FIG. 15 is a perspective view of the proximal end of the transducer of FIG. 15;

FIG. 16 is a perspective view of the transducer of FIG. 12;

FIG. 17 is a perspective view of the outer sheath of the surgical instrument of FIG. 11 including a portion of the movable interconnect electrically connected to the outer sheath;

FIG. 18 is an embodiment of a quick connect usable to establish electrical connection between the electrically conductive body of the rotatable electrical connector and the movable interconnect;

FIG. 19 is a side cross-sectional view of the surgical instrument of FIG. 9 showing the rotatable electrical connector electrically connecting the mail jack connector of transducer FIG. 11 and the outer sheath of the surgical instrument of FIG. 9;

FIG. 20 is perspective view of another embodiment of the rotatable electrical connector of the surgical instrument of FIG. 8 having a carriage and a set of electrical pin contacts;

FIG. 21 is a perspective view of the carriage of the rotatable electrical connector of FIG. 20;

FIG. 22 is a perspective view of the rotatable electrical connector of FIG. 20 in the process of being secured to the housing of the surgical instrument of FIG. 8;

FIG. 23 is a perspective view of the rotatable electrical connector of FIG. 20 positioned in direct contact with the outer shaft the surgical instrument of FIG. 8;

FIG. 24 is a perspective view of the rotatable electrical connector of FIG. 20 coupled to a connector collar for securing rotatable electrical connector to a rotation collar of the surgical instrument of FIG. 8;

FIG. 25 a perspective view of the rotatable electrical connector and connector collar of FIG. 24 coupled to a rotation collar of the surgical instrument of FIG. 8 and including a wire connector frame electrically connected to the electrical pin contacts;

FIG. 26 is a perspective view of another embodiment of the rotatable electrical connector of the surgical instrument of FIG. 8 including a pin collar secured to the outer sheath of the surgical instrument and an electrical pin contact attached to the pin collar;

FIG. 27 is a cross-sectional view of another embodiment of the rotatable electrical connector of the surgical instrument of FIG. 8 including an electrical pin contact having a rounded pin head;

FIG. 28 is a cross-sectional view of another embodiment of the electrical pin contact of FIG. 27 having a curved pin head including a curvature similar to the curvature of the outer sheath of the surgical instrument of FIG. 8;

FIG. 29 is cross-sectional view of another embodiment of the rotatable electrical connector of the surgical instrument of FIG. 8 including an inner bearing ring attached to the outer sheath of the surgical instrument, an outer bearing ring, and a set of electrically conductive ball bearings located between the inner and outer bearing rings;

FIG. 30 is side elevation view of another embodiment of the rotatable electrical connector of FIG. 29 in which the inner and outer bearing rings are embodied as cylindrical bushings to facilitate axial movement of the outer sheath of the surgical instrument of FIG. 8;

FIG. 31 is a side elevation view of another embodiment of the rotatable electrical connector of the surgical instrument of FIG. 8 embodied as a spring clamp configured to couple to the outer sheath of the surgical instrument;

FIG. 32 is a side elevation view of the spring clamp of FIG. 31 coupled to the outer sheath of the surgical instrument of FIG. 8;

FIG. 33 is a side elevation view of another embodiment of the spring clamp of FIG. 31 configured to couple to the outer sheath of the surgical instrument of FIG. 8;

FIG. 34 is a side elevation view of the spring clamp of FIG. 33 coupled to the outer sheath of the surgical instrument of FIG. 8;

FIG. 35 is a side elevation view of another embodiment of the spring clamp of FIG. 31 configured to couple to the outer sheath of the surgical instrument of FIG. 8;

FIG. 36 is a side elevation view of the spring clamp of FIG. 35 coupled to the outer sheath of the surgical instrument of FIG. 8;

FIG. 37 is a side elevation view of another embodiment of the spring clamp of FIG. 31 configured to couple to the outer sheath of the surgical instrument of FIG. 8;

FIG. 38 is a side elevation view of the spring clamp of FIG. 37 coupled to the outer sheath of the surgical instrument of FIG. 8;

FIG. 39 is a side elevation view of another embodiment of the spring clamp of FIG. 31 configured to couple to the outer sheath of the surgical instrument of FIG. 8;

FIG. 40 is a side elevation view of the spring clamp of FIG. 39 coupled to the outer sheath of the surgical instrument of FIG. 8;

FIG. 41 is a perspective view of another embodiment of the rotatable electrical connector of the surgical instrument of FIG. 8 embodied as an electrical clip configured to couple directly to the outer sheath of the surgical instrument or to a rotation collar of the surgical instrument of FIG. 8;

FIG. 42 is a perspective view of the electrical clip of FIG. 41 including a locking bracket configured to secure both ends of the electrical clip;

FIG. 43 is a perspective view of the electrical clip of FIG. 41 coupled to the outer sheath of the surgical instrument of FIG. 8;

FIG. 44 is a perspective view of the electrical clip of FIG. 41 coupled to the rotation collar of the surgical instrument of FIG. 8;

FIG. 45 is a perspective view of the electrical clip of FIG. 44 coupled to the rotation collar of the surgical instrument of FIG. 8 and including the locking bracket of FIG. 42; and

FIG. 46 is a simplified flow diagram of a method for monitoring operation of the surgical instrument of FIG. 8, which may be executed by the controller of FIG. 8.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient's natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (b); (c); (a and B); (a and C); (b and C); or (a, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

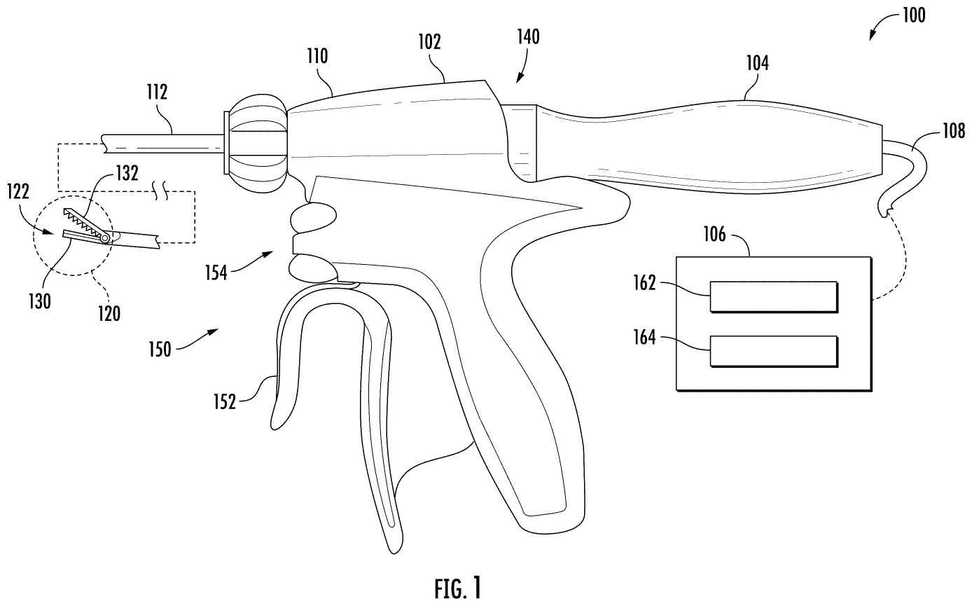

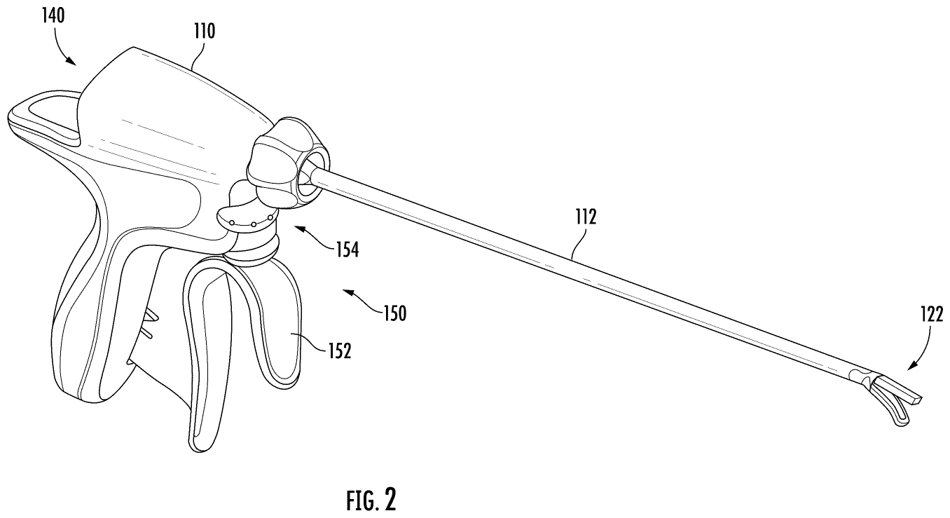

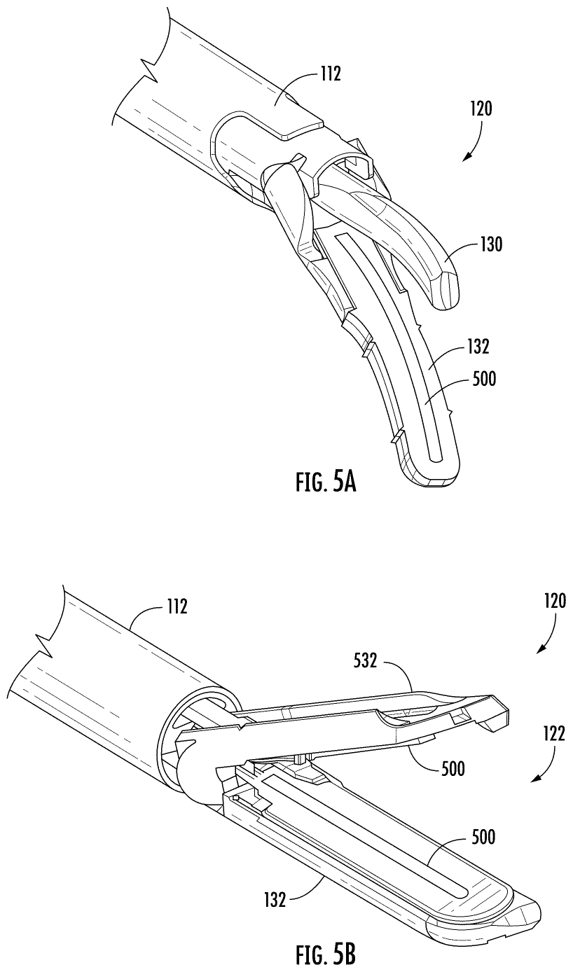

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

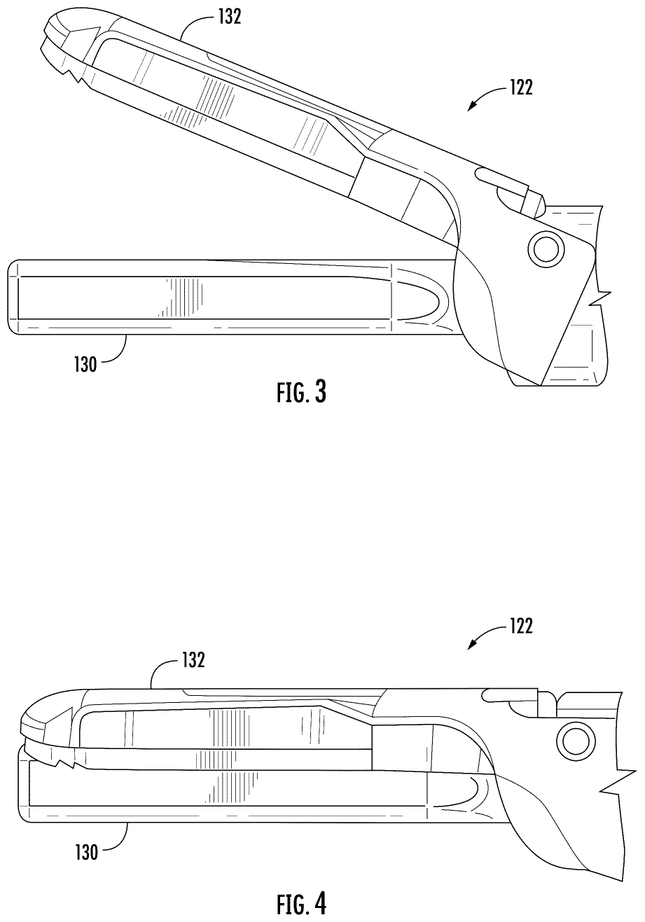

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both an ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient's body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient's tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient's tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient's tissue. For example, the electro-surgical generator module 164 may be configured to measure an impedance of the patient's tissue using the electrodes 500 and a suitable RF energy level.

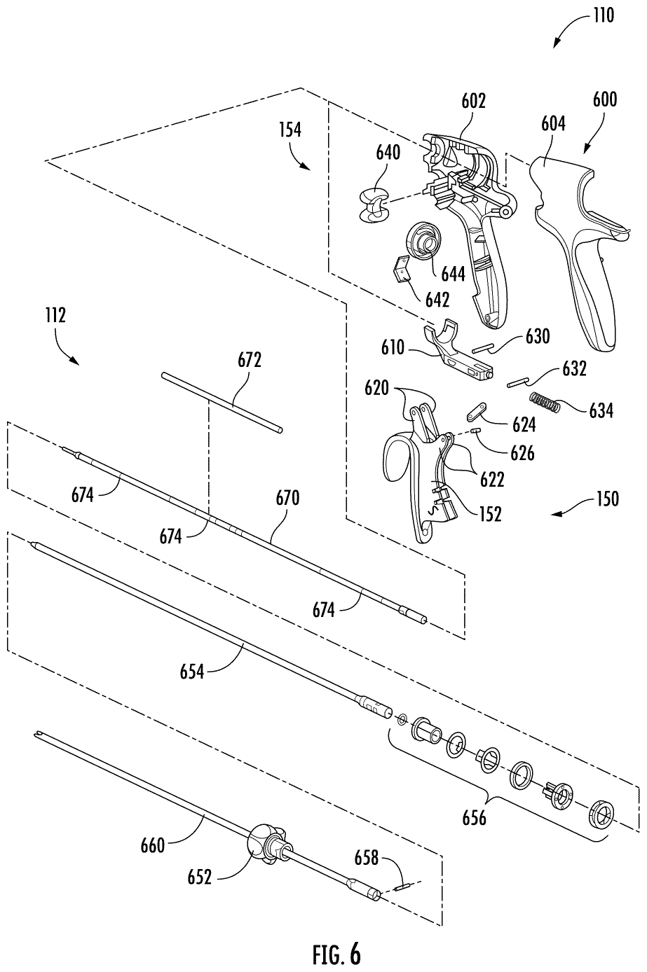

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the i/o subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

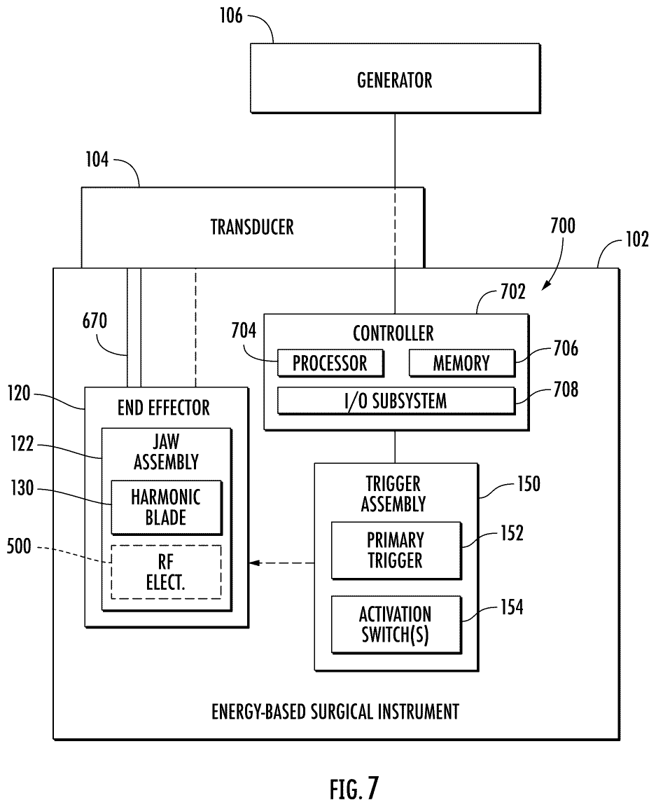

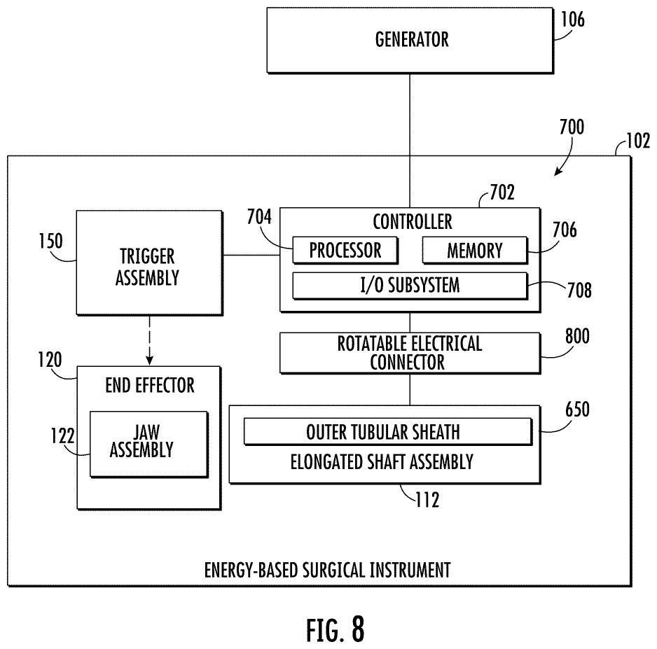

Referring now to FIG. 8, in another embodiment, the surgical instrument 102 includes a rotatable electrical connector 800. The rotatable electrical connector 800 provides an electrical connection between the outer tubular sheath 650 of the elongated shaft assembly 112 (see FIG. 2) and a power source of bipolar energy such as the controller 702 and/or the generator 106 to deliver the bipolar energy to the end effector. In this way, the outer tubular sheath 650 is used as an electrical conduit to provide the bipolar energy to the end effector 120 (e.g., to one or more electrodes of the end effector 120 for application to tissue captured by the jaw assembly 122 of the end effector 120). As described in more detail below, the rotatable electrical connector 800 is configured to maintain that electrical connection while facilitating rotation of the outer tubular sheath 650. To do so, the rotatable electric connector 800 maybe configured to rotate about the outer tubular sheath 650 and/or allow rotation of the outer tubular sheath 650, relative to the rotatable electrical connector 800, while maintaining the electrical connection between the outer tubular sheath and the power source of the bipolar energy.

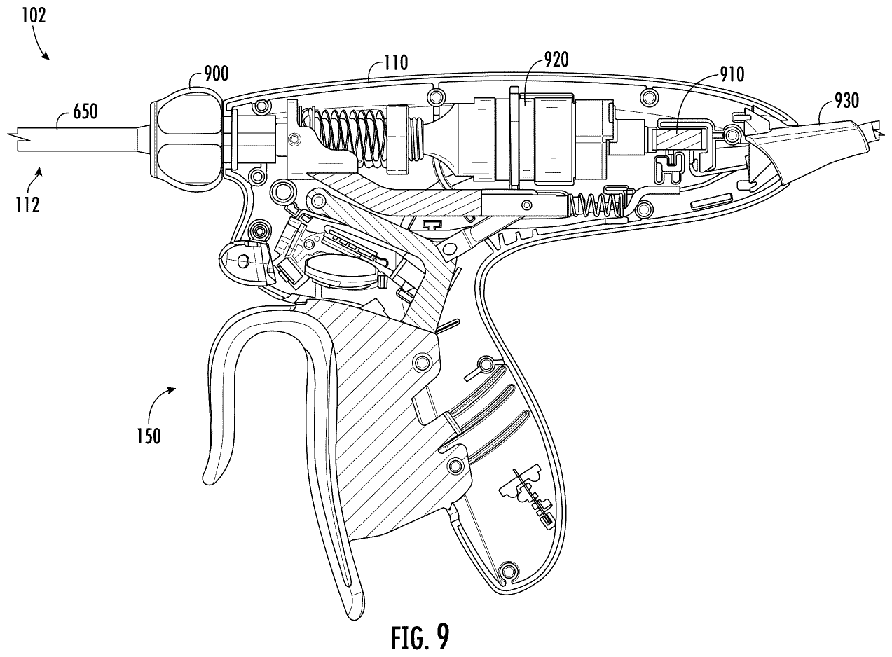

To facilitate rotation of the outer tubular sheath 650, the surgical instrument 102 may include a rotational knob 900 as shown in FIG. 9. The rotational knob 900 is configured to allow an operator of the surgical instrument 102 to manually rotate the outer tubular sheath 650 by manipulation of the rotational knob 900. Additionally, in some embodiments, the outer tubular sheath 650 may be linear or axially movable relative to the handle assembly 110.

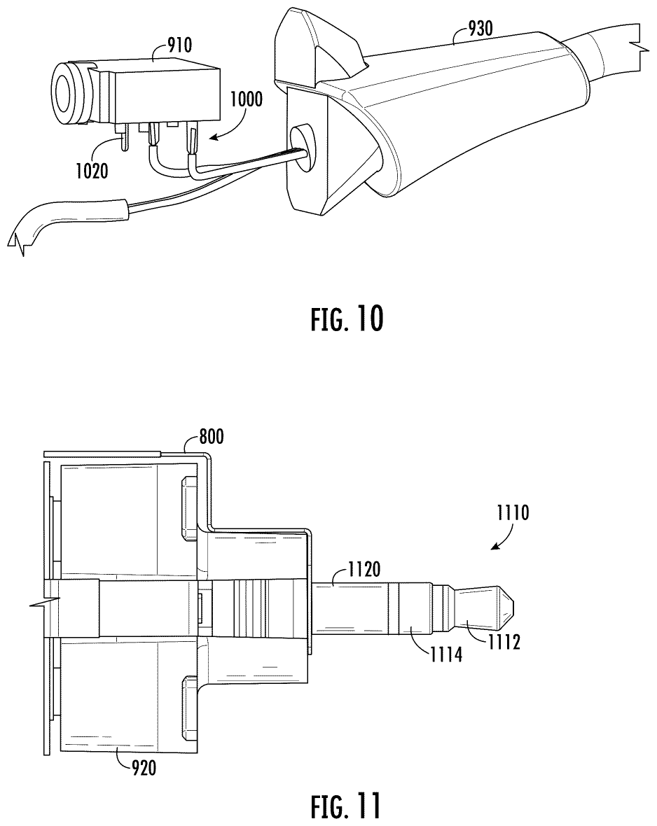

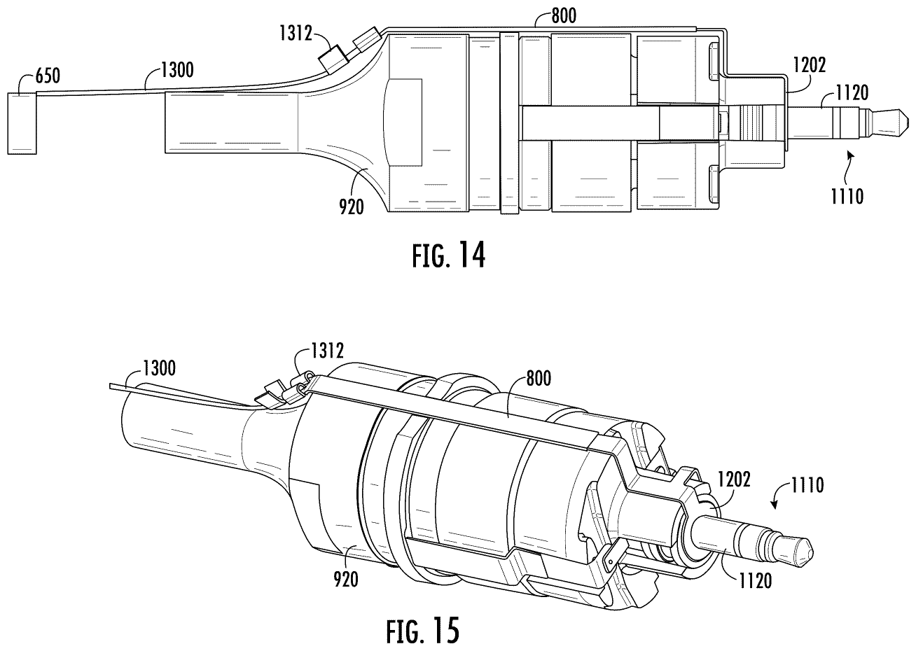

Referring now to FIGS. 9-19, in the illustrative embodiment of FIG. 9, the surgical instrument 102 includes a jack connector 910, which is configured to mate with a corresponding jack connector 1110 of a transducer 920 of the surgical instrument 102. The jack connector 910 is configured to provide energy to the transducer 920 to facilitate the generation of the ultrasonic energy during the harmonic mode of the surgical instrument 102. To do so, the jack connector 910 is electrically connected to an ultrasonic energy source (e.g., the generator 106) via a wire connector 930, which is attached to the handle assembly 110. As shown in FIG. 10, the wire connector 930 routes a set of wires, which are electrically connected to a pair of prongs 1000 of the jack connector 910. The illustrative jack connector 910, however, includes a third prong 1020, which is used to receive bipolar energy from a bipolar energy source (e.g., the generator 106 or the controller 702). As such, although not shown in FIG. 10 for clarity, the third prong 1020 is electrically connected to a wire of the wire connector 930 in the illustrative embodiment to supply the jack connector 910 with the bipolar energy.

As best shown in FIGS. 10 and 11, the jack connector 910 is configured to mate with the jack connector 1110 of the transducer 920 to supply the ultrasonic energy to the transducer 920. Illustratively, the jack connector 910 is embodied as a female jack connector and the jack connector 1110 is embodied as a male jack connector configured to mate with the jack connector 910. The jack connector 910 provides the ultrasonic energy to energy-receiving shaft portions 1112, 1114. Additionally, the jack connector 910 provides the bipolar energy to the energy-receiving shaft portion 1120 of the jack connector 1110.

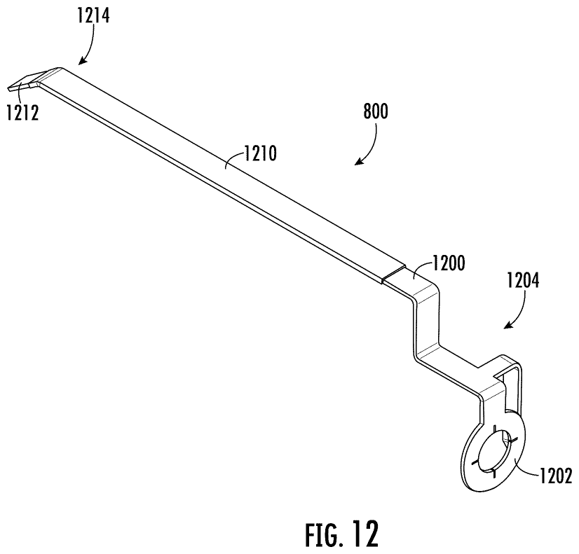

In the illustrative embodiment, the transducer 920 does not utilize the bipolar energy provided by the jack connector 910. However, the rotatable electrical connector 800 is electrically connected to the energy-receiving shaft portion 1120 of the jack connector 1110 to receive the bipolar energy and route the bipolar energy over the transducer 920 and to the outer tubular sheath 650 as described below. In the illustrative embodiment, as shown in FIG. 12, the rotatable electrical connector 800 includes an elongated electrically conductive body 1200 having an eyelet connector 1202 located at an end 1204 of the body 1200 and a tab connector 1212 at an opposite end 1214 of the body 1200. The elongated electrically conductive body 1200 may be formed from any suitable electrically conductive material such as copper. In some embodiments, rotatable electrical connector 800 may include an insulator covering 1220 positioned over the elongated electrically conductive body 1200 to provide an amount of electrical insulation. The insulator covering 1220 may be formed form any material capable of providing such electrical insulation such as a rubber material.

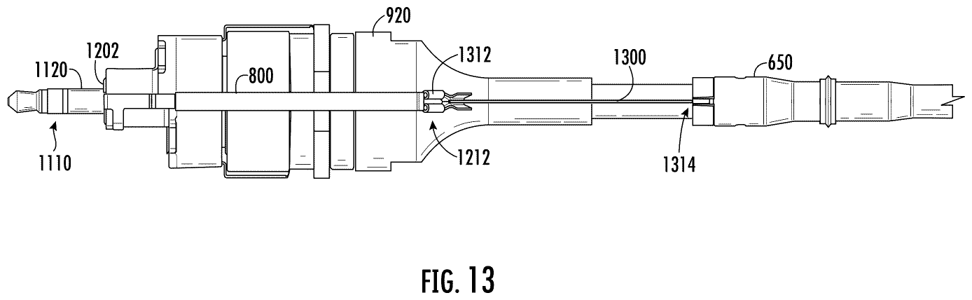

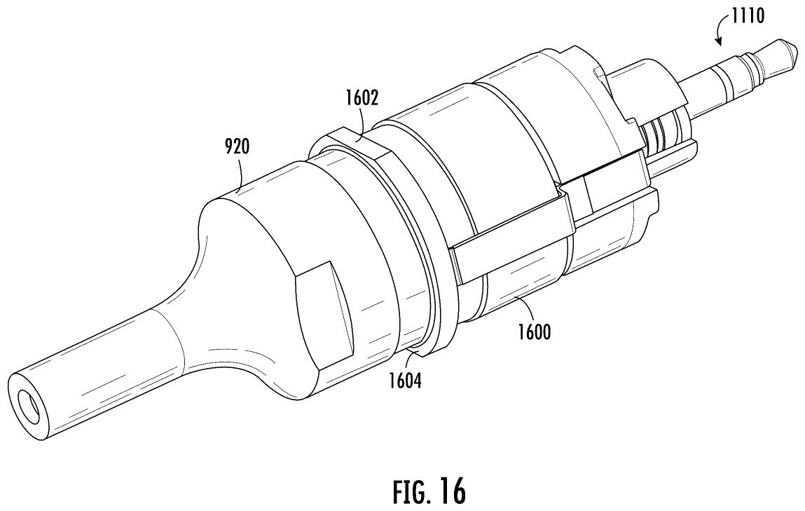

As shown in FIGS. 13-15, the rotatable electrical connector 800 is coupled to the transducer 920 such that the jack connector 1110 is received through the eyelet connector 1202 of the elongated electrically conductive body 1200. In such a position, the eyelet connector 1202 is in electrical contact with the energy-receiving shaft portion 1120 of the jack connector 1110 and the elongated electrically conductive body 1200 extends across the longitudinal length of the transducer 920. To facilitate the transversal of the elongated electrically conductive body 1200 across the transducer 920, the illustrative transducer 920 includes a body 1600 having a flat edge or surface 1602 across which the elongated electrically conductive body 1200 lies or otherwise transverses, as shown in FIG. 16. In some embodiments, the body 1600 of the transducer 920 may include another flat edge or surface 1604 located on an opposite side of the body 1600 from the flat edge 1602 to balance or improve the acoustic performance of the transducer 920.

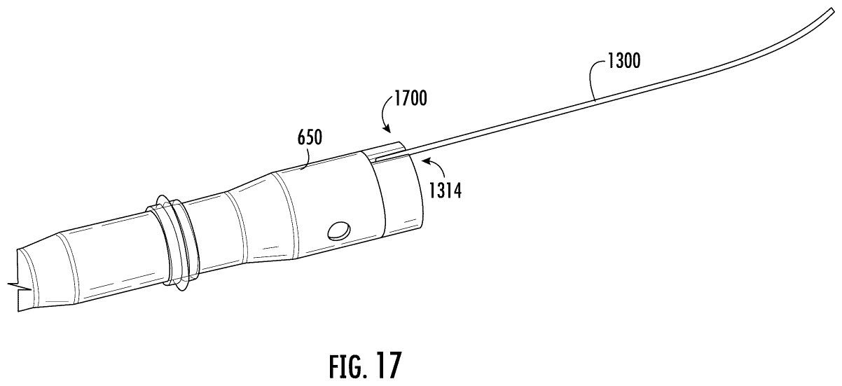

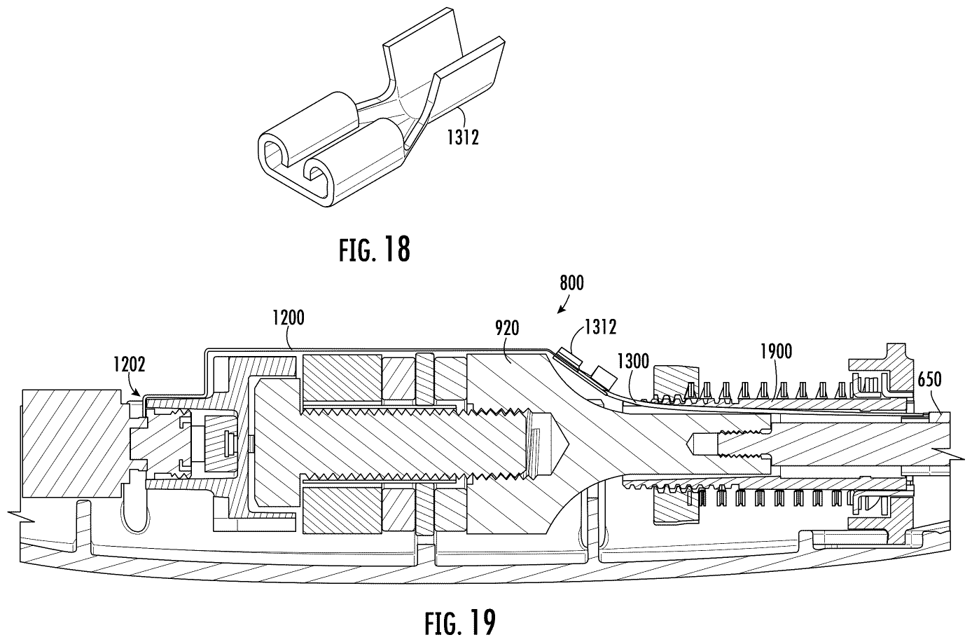

Again, as shown in FIGS. 13-15, the illustrative rotatable electrical connector 800 includes a movable or flexible electrical interconnect 1300, which is electrically coupled to the tab connector 1212 of the elongated electrically conductive body 1200 via an electrical connector 1312 (see FIG. 18). An end 1314 of the electrical interconnect 1300, opposite the electrical connector 1312, is electrically coupled to outer tubular sheath 650 via an electrical connection 1700 as shown in FIG. 17. The electrical interconnect 1300 is movable or otherwise flexible to allow an amount of misalignment between the blade 130 and the transducer 920. As shown in FIG. 19, the electrical interconnect 1300 may extend under or through other components of the surgical instrument 102 to facilitate the electrical connection to the outer tubular sheath 650.

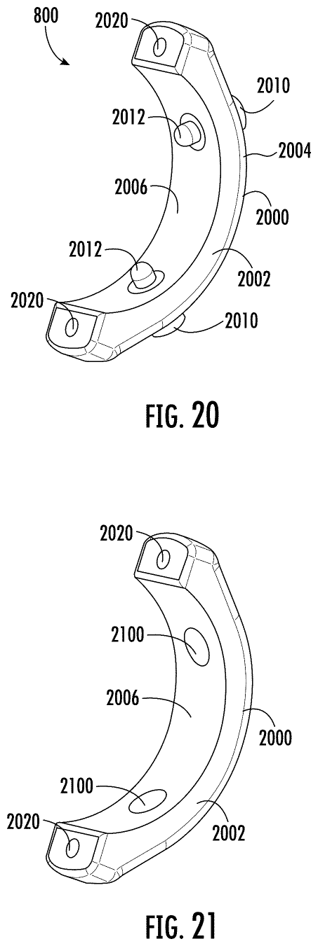

Referring now to FIGS. 20-25, in some embodiments, the rotatable electrical connector 800 may be embodied as, or otherwise include, a carriage 2000 and a set of electrically conductive pin contacts 2010. The carriage 2000 includes a carriage body 2002 having a set of apertures or passageways 2100 (see FIG. 21) that extend through the body from an outer surface 2004 to an inner surface 2006. Each electrically conductive pin contacts 2010 is received in a corresponding one of the passageways 2100 such that a pin head 2012 of each electrically conductive pin contact 2010 extends from the inner surface 2006. As discussed in more detail below, each of the pin heads 2012 are positioned and configured to electrically contact the outer tubular sheath 650 when the carriage 2000 is coupled to the outer tubular sheath 650 to thereby establish an electrical connection. Illustratively, the electrically conductive pin contacts 2010 are embodied as pogo-style pin in which the pin head 2012 is configured to be depressed into the body of the corresponding pin contact 2010 when the pin head 2012 is contacted with the outer tubular sheath 650.

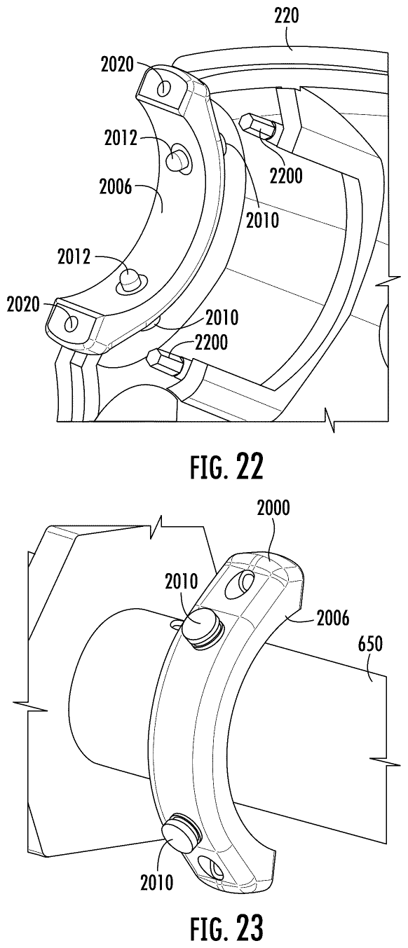

As shown in FIG. 22, the carriage 2000 is configured to be mounted to the handle assembly 110. To do so, the handle assembly 110 may include a pair of mounting pins 2200 that are configured to be received in mounting holes 2020 of the carriage 2000 to mount the carriage 2000 to the housing 110. As shown in FIG. 23, the carriage 2000 is mounted to the handle assembly 110 such that the carriage 2000 partially wraps around the outer tubular sheath 650. When so mounted, the pin heads 2012 of the electrically conductive pin contacts 2010 contact the outer tubular sheath 650 to establish an electrical connection therewith. To facilitate proper coupling of the carriage 2000 to the outer tubular sheath 650, the inner surface 2006 has a concave curvature that matches the convex curvature of the outer tubular sheath 650 sufficiently enough to allow proper positioning of the pin heads 2012 on the outer tubular sheath 650.

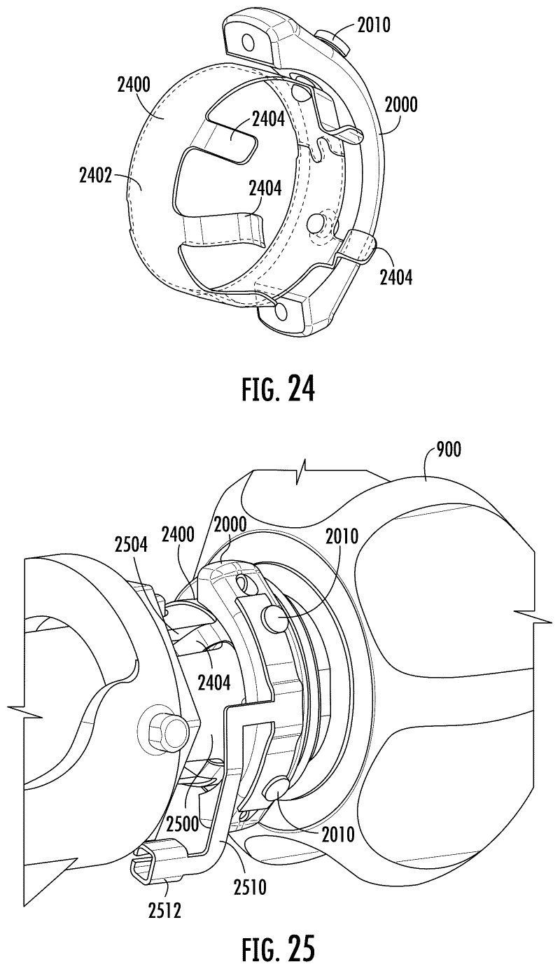

Referring now to FIGS. 24 and 25, in some embodiments, the rotatable electrical connector 800 may include a connector collar 2400 configured to be used with the carriage 2000. The connector collar 2400 is electrically conductive and includes a ring body 2402 having a set of tabs 2404 that extend axially inwardly. The ring body 2402 is shaped and configured to be received within the carriage 2000 as shown in FIG. 24. When so received, each of the pin heads 2012 of the electrically conductive pin contacts 2010 contacts the ring body 2402 of the connector collar 2400.

As shown in FIG. 25, the connector collar 2400 is configured to be attached to a rotation collar 2500 of the surgical instrument. In such embodiments, the rotation collar surrounds the outer tubular sheath 650 and is formed from a non-conductive material. As such, the rotation collar 2500 includes a set of apertures 2504 that provides access to the outer tubular sheath 650. When the connector collar 2400 and the carriage 2000 are coupled to the outer tubular sheath 650 as shown in FIG. 25, each tab 2404 of the connector collar 2400 extends into a corresponding aperture 2504 of the rotation collar 2500 and is positioned in contact with the outer tubular sheath 650. The illustrative rotatable electrical connector 800 may also includes a wire connector frame 2510 which is coupled to the carriage 2000 via the electrically conductive pin contacts 2010. The wire connector frame 2510 includes wire connector 2512 to which a wire carrying the bipolar energy may be coupled.

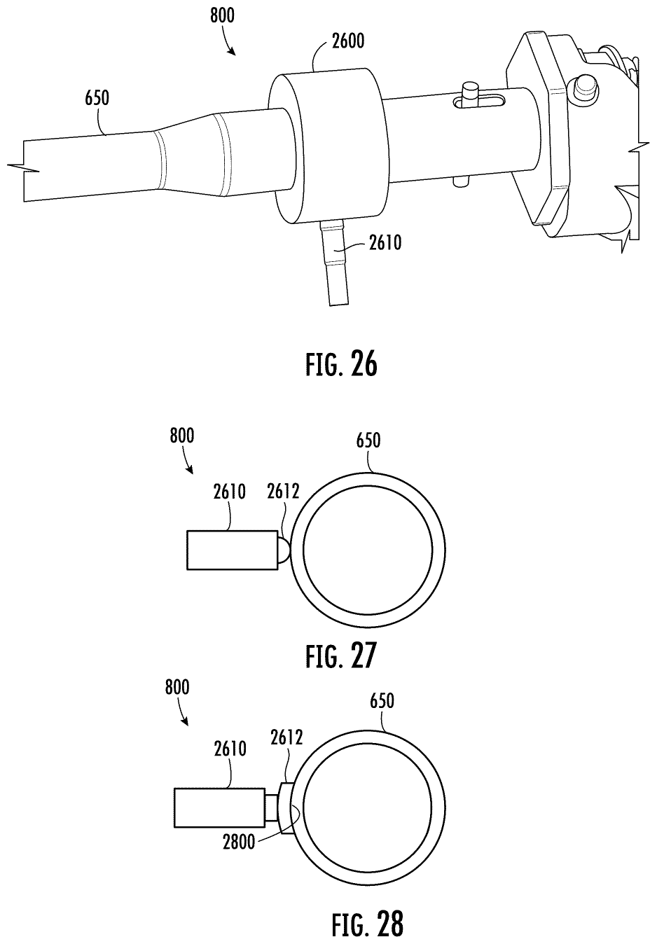

Referring now to FIGS. 26-28, in some embodiments, the rotatable electrical connector 800 may be embodied as, or otherwise include, a cylindrical pin collar 2600 configured to be coupled to and encircle the outer tubular sheath 650. The cylindrical pin collar 2600 includes a set of electrically conductive pins 2602 that extend through the cylindrical pin collar 2600 such that a pin head 2612 (see FIGS. 27 and 28) of each electrically conductive pin 2602 is in contact with the outer tubular sheath 650. In some embodiments, as shown in FIG. 27, each pin head 2612 of the electrically conductive pins 2602 may have a rounded or circular cross-section. Alternatively, in other embodiments, as shown in FIG. 28, each pin head 2612 of the electrically conductive pins 2602 may have a concave contact surface 2800 having a curvature configured to match the convex curvature of the outer tubular sheath 650 such that the concave contact surface 2800 is fully engaged with the outer tubular sheath 650.

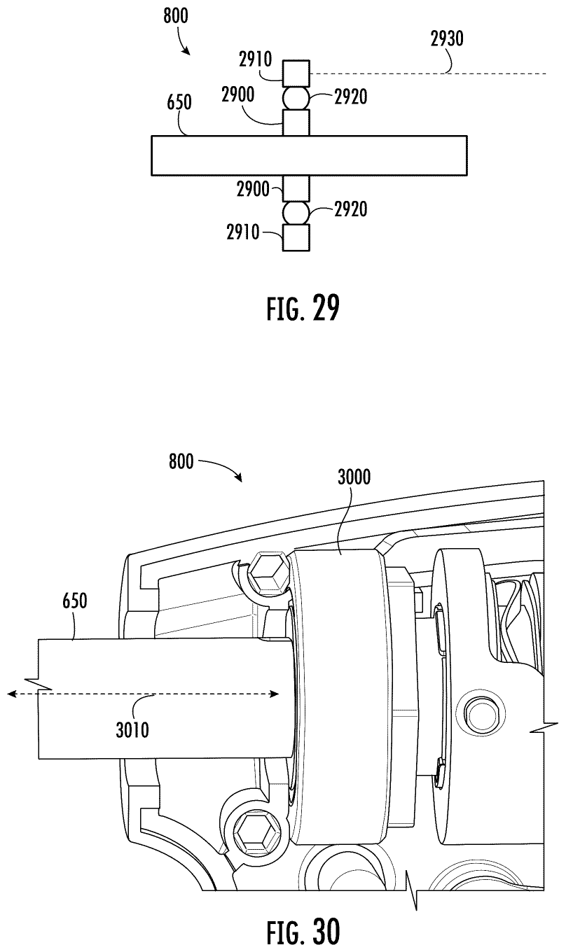

Referring now to FIGS. 29 and 30, in some embodiments, the rotatable electrical connector 800 may be embodied as, or otherwise include, an inner bearing ring 2900 coupled to the outer tubular sheath 650 and a outer bearing ring 2910. Each of the inner bearing ring 2900 and the outer bearing ring 2910 is electrically conductive. Additionally, a set of electrically conductive ball bearings are sandwiched between the inner bearing ring 2900 and the outer bearing ring 2910. An electrical interconnect 2930 may be electrically connected to the outer bearing ring 2910 to provide the bipolar energy to the rotatable electrical connector 800. Because each of the outer bearing ring 2910, the inner bearing ring 2900, and the set of ball bearings 2920 are electrically conductive, an electrical connection is established to transfer the bipolar energy received from the electrical interconnect 2930 to the outer tubular sheath 650. Additionally, the outer bearing ring 2910, the inner bearing ring 2900, and the set of ball bearings 2920 are configured to allow rotation to the outer tubular sheath while maintaining the electrical connection to transfer the bipolar energy to the outer tubular sheath 650. Furthermore, in some embodiments as shown in FIG. 30, the outer bearing ring 2910 and the inner bearing ring 2900 may be embodied as cylindrical bearing rings 3000 that are configured to facilitate distal-proximal axial movement of the outer tubular sheath along a rotational axis 3010.

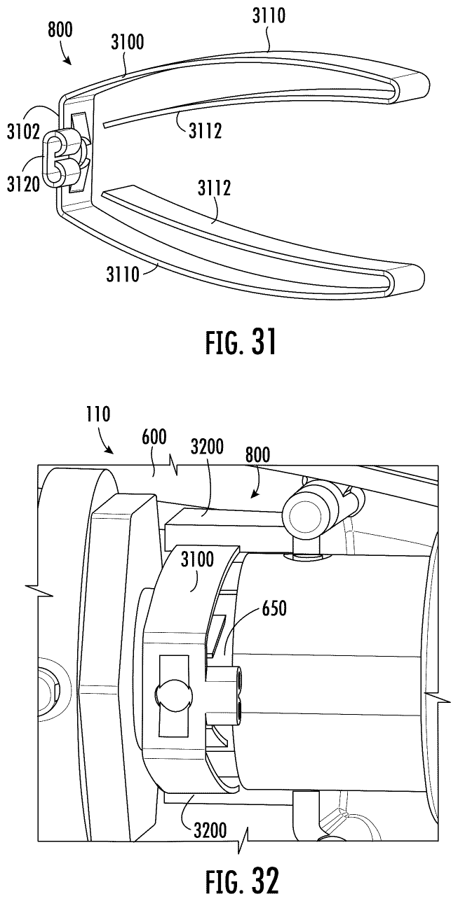

Referring now to FIGS. 31-40, in some embodiments, the rotatable electrical connector 800 may be embodied as, or otherwise include, a spring clamp configured to couple to the outer tubular sheath 650 such that the spring clamp is in electrical contact with the outer tubular sheath 650. In so doing, the spring clamp allows rotation of the outer tubular sheath 650 while maintaining the electrical contact. For example, an embodiment of a spring claim 3100 is shown in FIG. 31. The spring clamp 3100 includes a body 3102 and a pair of arms 3110. Illustratively, each arm 3110 includes a corresponding arm branch 3112 that extends backwardly toward the body 3102. The arm branches 3112 are configured to depress outwardly toward the corresponding arm 3110 when the outer tubular sheath 650 is being received within the spring clamp 3100 and exert pressure inwardly once the outer tubular sheath 650 is fully received in the spring clamp 3100 to thereby secure the spring clamp 3100 to the outer tubular sheath 650 as shown in FIG. 32. Additionally, in some embodiments, the housing 600 of the handle assembly 110 may include one or more ribs 3200 configured to press the arms 3110 inwardly toward the outer tubular sheath 650 to further secure the spring clamp 3100 to the outer tubular sheath 650. The spring clamp 3100 may also include a wire connector 3120 to which a wire carrying the bipolar energy may be coupled.

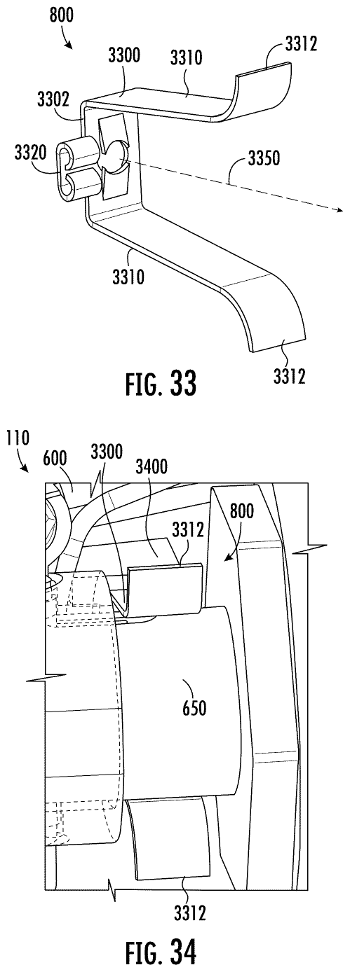

In other embodiments, as shown in FIGS. 33 and 34, the spring clamp may be embodied as a spring clamp 3300 including a body 3302 and a pair of arms 3310. Illustratively, each arm 3310 includes an outwardly flared end 3312, which extend away from a center axis 3350 of the spring clamp 3300. The flared ends 3312 facilitate receival of the outer tubular sheath 650 into the spring clamp 3300. Additionally, in some embodiments as shown in FIG. 34, the housing 600 of the handle assembly 110 may include one or more ribs 3400 configured to press the arms 3310 inwardly toward the outer tubular sheath 650 to further secure the spring clamp 3300 to the outer tubular sheath 650. The spring clamp 3300 may also include a wire connector 3320 to which a wire carrying the bipolar energy may be coupled.

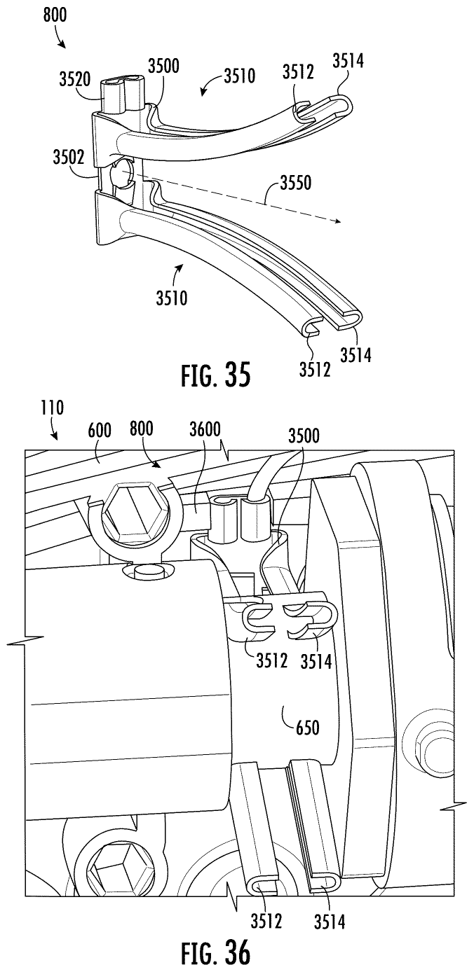

Referring now to FIGS. 35 and 36, in other embodiments, the spring clamp may be embodied as a spring clamp 3500 including a body 3502 and pair of arms 3510. Each arm 3510 includes separated arm branches 3512, 3514 which extend from the body 3502 and curve upwardly from a center axis 3550 of the spring clam 3500. The curvature of the arm branches 3512, 3514 provide additional structural strength. Additionally each arm branch 3512, 3514 is wrapped around to define a “U” shaped cross-sectional profile to provide further strength to the arms 3510. The curvature of the arm branches 3512, 3514 also facilitate receival of the outer tubular sheath 650 into the spring clamp 3500. Furthermore, in some embodiments as shown in FIG. 36, the housing 600 of the handle assembly 110 may include one or more ribs 3800 configured to press the arm branches 3512, 3514 inwardly toward the outer tubular sheath 650 to further secure the spring clamp 3500 to the outer tubular sheath 650. The spring clamp 3500 may also include a wire connector 3520 to which a wire carrying the bipolar energy may be coupled.

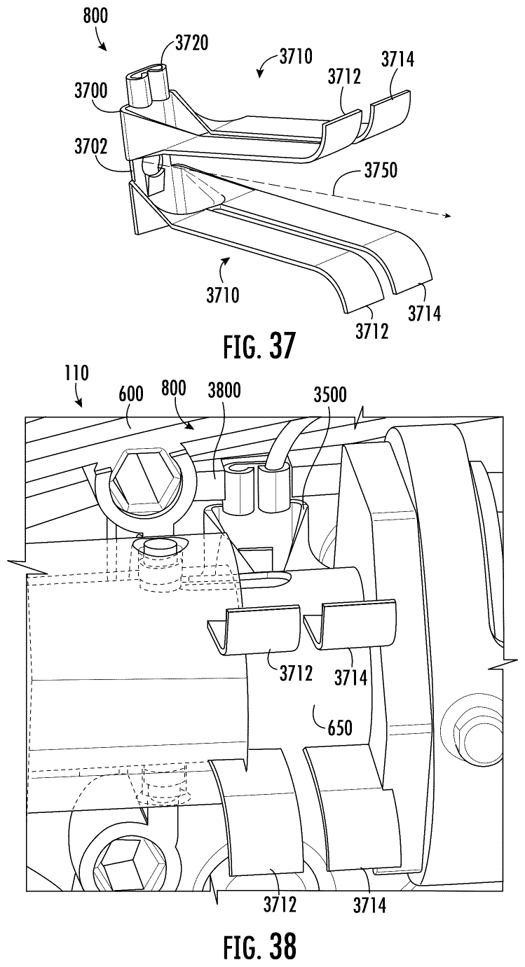

In other embodiments, as shown in FIGS. 37 and 38, the spring clamp may be embodied as a spring clamp 3700 including a body 3702 and a pair of arms 3710. Each arm 3710 includes separated arm branches 3712, 3714 which extend from the body 3702 and curve upwardly from a center axis 3750 of the spring clam 3700. Additionally, the distal ends of each arm branch 3712, 3714 is further flared outwardly to facilitate receival of the outer tubular sheath 650 into the spring clamp 3700. Additionally, in some embodiments as shown in FIG. 38, the housing 600 of the handle assembly 110 may include one or more ribs 3800 configured to press the arm branches 3712, 3714 inwardly toward the outer tubular sheath 650 to further secure the spring clamp 3700 to the outer tubular sheath 650. The spring clamp 3700 may also include a wire connector 3720 to which a wire carrying the bipolar energy may be coupled.

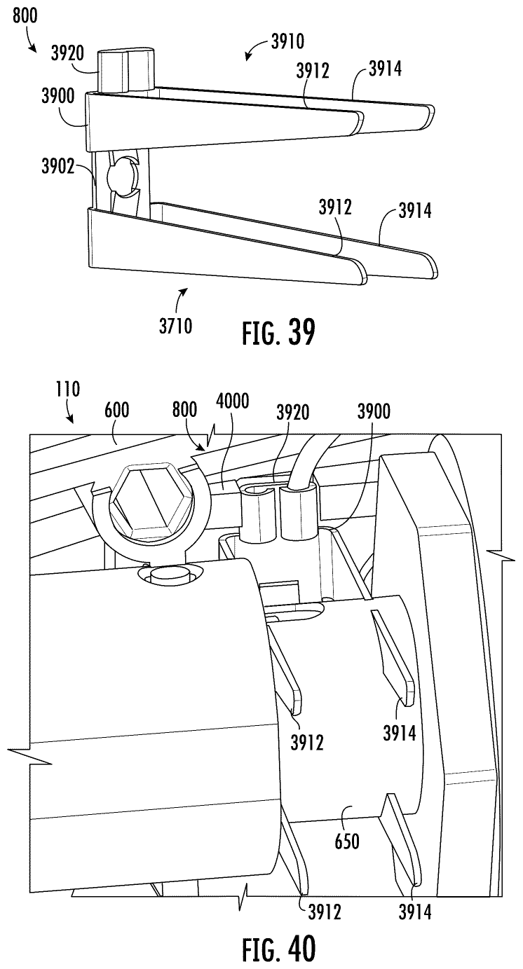

Further, as shown in FIGS. 39 and 40, the spring clamp may be embodied as a spring clamp 3900 in other embodiments. The spring clamp 3900 includes a body 3902 and a pair of arms 3910. Each arm 3910 includes separated arm branches 3912, 3914 which extend from the body 3902 and have a flat, triangular side profile. In this way, the arm branches 3912, 1394 have a general “blade” shape configured to facilitate receival of the outer tubular sheath 650 into the spring clamp 3900. Additionally, in some embodiments as shown in FIG. 40, the housing 600 of the handle assembly 110 may include one or more ribs 4000 configured to press the arm branches 3912, 3914 inwardly toward the outer tubular sheath 650 to further secure the spring clamp 3700 to the outer tubular sheath 650. The spring clamp 3900 may also include a wire connector 3920 to which a wire carrying the bipolar energy may be coupled.

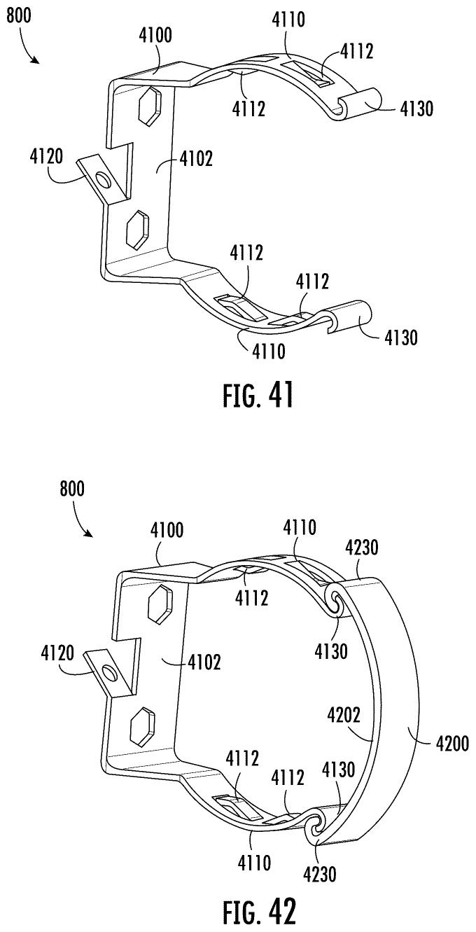

Referring now to FIGS. 41-45, in another embodiment, the rotatable electrical connector 800 may be embodied as, or otherwise include, an electrically conductive clip 4100 configured to be coupled, and partially encircle, the outer tubular sheath 650. the electrically conductive clip 4100 is formed from an electrically conductive material, such as an electrically conductive metallic material, and illustrative includes a flat body 4102 and a pair of curved arms 4110 extending from the flat body 4102. The curved arms 4110 form a partial circle having a diameter that matches or is otherwise similar to the diameter of the outer tubular sheath 650 to facilitate attachment of the electrically conductive clip 4100 to the outer tubular sheath 650.

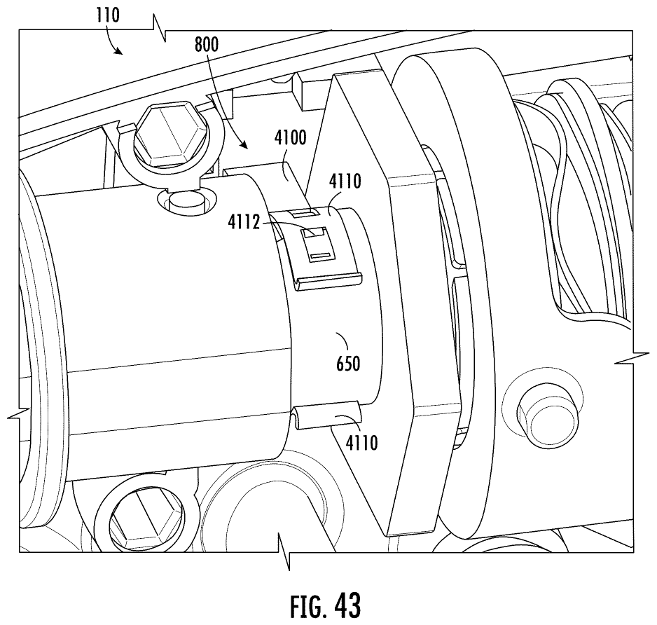

Each curved arm 4110 of the electrically conductive clip 4100 includes a detent 4112 that extends inwardly from the corresponding curved arm 4110. The detents 4112 are each configured to contact the outer tubular sheath 650 when the outer tubular sheath 650 is received within the electrically conductive clip 4100 to establish an electrical connection as shown in FIG. 43. Although the electrically conductive clip 4100 is shown as including only four detents 4112, the electrically conductive clip 4100 may include additional or fewer detents 4112 in other embodiments. Additionally, in some embodiments, the rotatable electrical connector 800 may also include a locking bracket 4200 configured to attach to ach of the curved arms 4110 of the electrically conductive clip 4100 as shown in FIG. 42. The locking bracket 4200 has a curved body 4202 that further defines a circular side profile of the rotatable electrical connector 800 when the locking bracket 4200 is attached to the electrically conductive clip 4100. To do so, the curved body 4202 of the locking bracket 4200 includes a pair of a curved ends 4230, each of which is configured to mate with a corresponding curved end 4130 of the curved arms 4110 of the electrically conductive clip 4100. The electrically conductive clip 4100 may also include a wire connector 4120 to which a wire carrying the bipolar energy may be coupled.

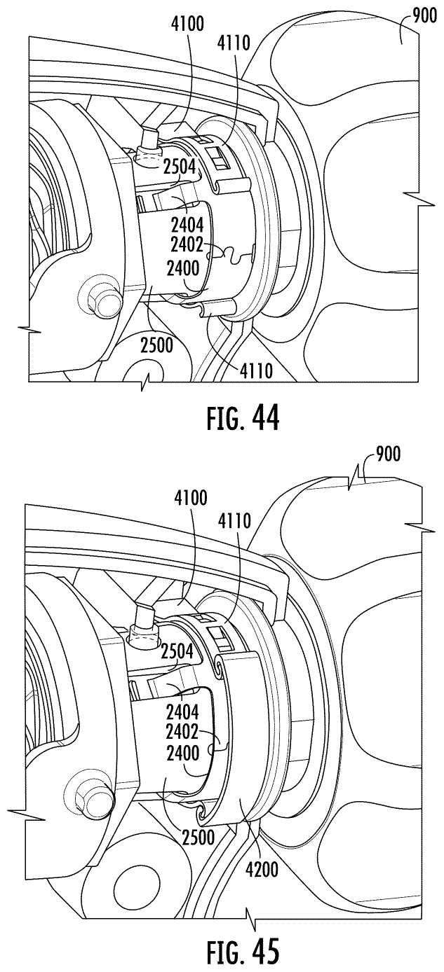

Similar to the embodiment of FIGS. 24 and 25, the rotatable electrical connector 800 may also include the connector collar 2400 configured to be used with the electrically conductive clip 4100 as shown in FIGS. 44 and 45. Again, the connector collar 2400 is electrically conductive and includes the ring body 2402 having the set of tabs 2404 that extend axially inwardly. The ring body 2402 is shaped and configured to be received within the electrically conductive clip 4100 as shown in FIG. 44. When so received, each of the detents 4112 of the electrically conductive clip 4100 contacts the ring body 2402 of the connector collar 2400.

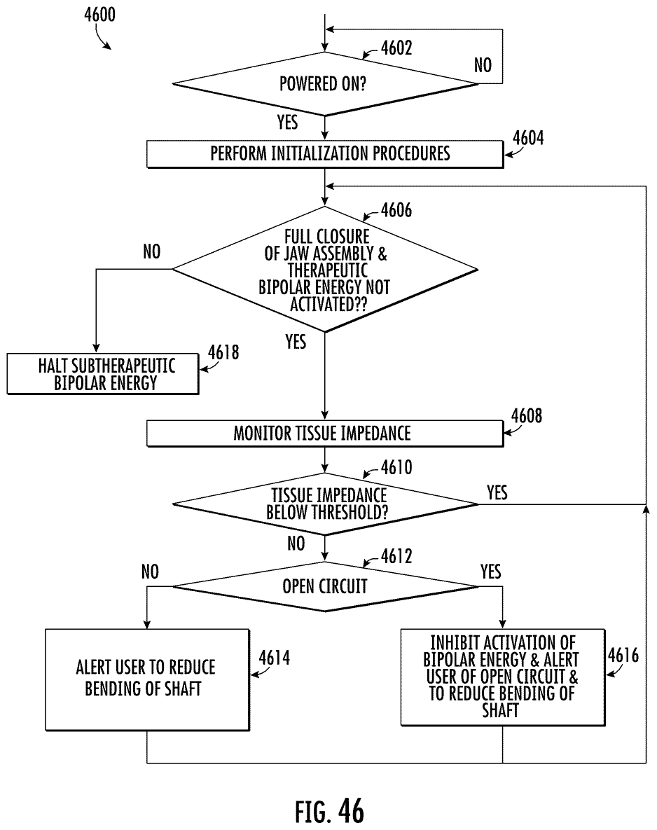

As shown in FIGS. 45 and 46 and as discussed above, the connector collar 2400 is configured to be attached to the rotation collar 2500 of the surgical instrument 102. Again, as described above, the rotation collar 2500 surrounds the outer tubular sheath 650 and is formed from a non-conductive material. As such, the rotation collar 2500 includes the set of apertures 2504 that provides access to the outer tubular sheath 650. when the connector collar 2400 and the electrically conductive clip 4100 are coupled to the outer tubular sheath 650 as shown in FIGS. 45 and 46, each tab 2404 of the connector collar 2400 extends into a corresponding aperture 2504 of the rotation collar 2500 and is positioned in contact with the outer tubular sheath 650. In some embodiments, as shown in FIG. 46, the locking bracket 4200 may be attached the curved arms 4110 of the electrically conductive clip 4100 to secure the electrically conductive clip 4100 to the outer tubular sheath 650.

Referring now to FIG. 46, in certain situations, an operator of the surgical instrument 102 may inadvertently bend or otherwise apply significant pressure to the elongated shaft assembly 112, which can cause an increase in RF impedance and/or otherwise result in poor performance of the application of bipolar energy to tissue captured in the jaw assembly 122 of the surgical instrument. As such, in some embodiment, the controller 702 (and/or generator 106) may be configured to execute a method 4600 for monitoring operation of the surgical instrument 102 to reduce the occurrence of deformation of the elongated shaft assembly 112.

The method 4600 begins with block 4602 in which in which the controller 702 determines whether the energy-based surgical instrument 102 has been powered to an “on” state. If so, the method 4600 advances to block 4604 in which the controller 702 performs one or more initialization procedures. For example, the controller 702 may verify electrical connection of the rotatable electrical connector 800, connection of the transducer 104 and/or remote generator 106, and/or other initialization or verification procedure.

After the controller 702 has performed the initialization procedures in block 4604, the method 4600 advances to block 4606 in which the controller 702 determines whether the jaw assembly 122 is in a fully closed state and whether a therapeutic level of bipolar energy has not yet been activated. The jaw assembly may be moved to the fully closed state, for example, to grasp tissue. If jaw assembly 122 is in the fully closed state and the bipolar energy has not yet been activated, the method 4600 advances to block 4608. In block 4608, the controller 702 monitors the impedance of tissue captured in the jaw assembly 122. To do so, the controller 702 may apply a sub-therapeutic level of bipolar energy to the tissue and measure a response caused by the tissue (e.g., a change in voltage or current). Subsequently, in block 4610, the controller 720 compares the measured tissue impedance to a reference impedance threshold. For example, in an illustrative embodiment, the reference impedance threshold may be set to about 20 ohms.

If the controller 702 determines that the measured impedance is below the reference impedance threshold, the method 4600 loops back to block 4606 in which the controller 702 again determines whether the jaw assembly 122 is in a fully closed state and whether a therapeutic level of bipolar energy has not yet been activated. If, however, the measured impedance is not below the reference impedance threshold, the method 4600 advances to block 4612. In block 4612, the controller 702 determines if the measured impedance is indicative of an open circuit. To do so, for example, the controller 702 may compare the measured impedance to a second, higher reference impedance. If the measured impedance is not indicative of an open circuit, the method 4600 advances to block 4614 in which the controller 702 (or the generator 106) alerts the user to reduce the bending or deformation of the elongated shaft assembly 112. For example, the controller 702 may generate an audible, visual, or tactile alert to the user.

However, if the measured impedance is indicative of an open circuit in block 4612, the method 4600 advances to block 4616. In block 4616, the controller 702 (or generator 106) inhibits the activation of the bipolar energy of the surgical instrument 102. Additionally, the controller 702 (or generator 106) alerts the user of the detected “open circuit” and requests reduction of any bending or deformation of the elongated shaft assembly 112. Again, the controller 702 may generate an audible, visual, or tactile alert to the user. The method 4600 subsequently loops back to block 4606 in which the controller 702 again determines whether the jaw assembly 122 is in a fully closed state and whether a therapeutic level of bipolar energy has not yet been activated. Referring back to block 4606, if the jaw assembly 122 is not in the fully closed state and/or the therapeutic bipolar energy has been activated, the method 4600 advances to block 4618. In block 4618, the controller 702 (or generator 106) halts the application of the therapeutic bipolar energy and discontinues the measurement of the tissue impedance.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A surgical instrument for performing an energy-based surgical procedure, the surgical instrument comprising:
an elongated shaft assembly having an outer tubular sheath and an end effector having a jaw assembly movable between an open state and a closed state, wherein the jaw assembly includes an ultrasonic blade;
a trigger assembly operable to move the jaw assembly of the end effector between the open state and the closed state and to apply bipolar energy to tissue captured within the jaw assembly; and
a rotatable electrical connector configured to establish an electrical connection between the outer tubular sheath and an energy source of the bipolar energy, wherein the rotatable electrical connector is configured to facilitate rotation of the outer tubular sheath while maintaining the electrical connection between the outer tubular sheath and the energy source.

2. The surgical instrument of claim 1, wherein the rotatable electrical connector comprises an elongated electrically conductive body having an eyelet connector at a first end and a tab connector at a second end opposite the first end, wherein the eyelet connector is configured to receive a male jack connector extending from a proximal end of a transducer of the surgical instrument and wherein the eyelet connector is configured to rotate about the male jack connector.

3. The surgical instrument of claim 2, wherein the rotatable electrical connector further includes an electrical interconnect having a first end electrically connected to the outer tubular sheath of the elongated shaft assembly and a second end, opposite the first end, electrically connected to a connector configured to mate with the tab connector of the elongated electrically conductive body, wherein the elongated electrically conductive body and the electrical interconnect cooperate to establish and electrical connection between the male jack connector of the transducer and the outer tubular sheath.

4. The surgical instrument of claim 1, wherein the rotatable electrical connector comprises a carriage and a set of electrical pin contacts extending through carriage, wherein the carriage is coupled to a housing of the surgical instrument and each electrical pin contact includes an electrically conductive pinhead in contact with the outer tubular sheath.

5. The surgical instrument of claim 1, wherein the rotatable electrical connector comprises an electrically conductive connector collar configured to couple with a electrically non-conductive rotation collar coupled to the outer tubular sheath, a carriage configured to couple to the electrically conductive connector collar, and a set of electrical pin contacts extending through carriage,
wherein the electrically non-conductive rotation collar includes a plurality of apertures and wherein the electrically conductive connector collar includes a plurality of tabs that extend axially inwardly, wherein each tab of the plurality of tabs extends into a corresponding aperture of the plurality of apertures and is in contact with the outer tubular sheath,
wherein each electrical pin contact includes an electrically conductive pinhead in contact with the electrically conductive connector collar.

6. The surgical instrument of claim 5, wherein the rotatable electrical connector further includes a wire connector frame electrically coupled to each electrical pin contact and including a connector configured for electrical connection to an interconnect.

7. The surgical instrument of claim 1, wherein the rotatable electrical connector comprises a cylindrical pin collar coupled to and encircling the outer tubular sheath and a set of electrical pin contacts extending through the cylindrical pin collar, wherein each electrical pin contact includes an electrically conductive pinhead in contact with the outer tubular sheath.

8. The surgical instrument of claim 7, wherein the electrically conductive pinhead of each electrical pin contact is rounded.

9. The surgical instrument of claim 7, wherein the electrically conductive pinhead of each electrical pin contact has a concave surface having a curvature that matches a curvature of an outside surface of the outer tubular sheath.

10. The surgical instrument of claim 1, wherein the rotatable electrical connector comprises an electrically conductive inner bearing ring attached to the outer tubular sheath, an electrically conductive outer bearing ring encircling the inner bearing ring, and a set of electrically conductive ball bearings positioned between the electrically conductive inner bearing ring and the electrically conductive outer bearing ring.

11. The surgical instrument of claim 10, wherein the rotatable electrical connector further comprises an electrical interconnect electrically connected to the electrically conductive outer bearing ring.

12. The surgical instrument of claim 10, wherein each of the electrically conductive inner bearing ring and the electrically conductive outer bearing ring comprise electrically conductive cylindrical bushings configured to facilitate axial movement of the outer tubular sheath through the rotatable electrical connector.

13. The surgical instrument of claim 1, wherein the rotatable electrical connector comprises an electrically conductive spring clamp having a first arm extending around a first section of the outer tubular sheath and a second arm, opposite the first arm, extending around a second section of the outer tubular sheath opposite the first section.

14. The surgical instrument of claim 13, wherein each arm of the electrically conductive spring clamp includes an arm branch that extends axially inwardly and is configured to be deflected axially outwardly to facilitate connection of the electrically conductive spring clamp to the outer tubular sheath.

15. The surgical instrument of claim 13, wherein each arm of the electrically conductive spring clamp curves away from the other arm.

16. The surgical instrument of claim 13, wherein each arm of the electrically conductive spring clamp includes a first arm branch and a second arm branch spaced from the first arm branch.

17. The surgical instrument of claim 1, wherein the rotatable electrical connector comprises an electrically conductive clip configured to attach to the outer tubular sheath, wherein the electrically conductive clip includes a first arm extending around a first section of the outer tubular sheath and a second arm, opposite the first arm, extending around a second section of the outer tubular sheath opposite the first section, and wherein each arm includes at least one detent that extends axially inwardly and is configured to contact the outer tubular sheath when the electrically conductive clip is attached to the outer tubular sheath.

18. The surgical instrument of claim 17, wherein the electrically conductive clip further includes a locking bracket configured to attach to each of the first arm and the second arm to lock the electrically conductive clip to the outer tubular sheath.

19. A method for monitoring operation of a surgical instrument, the method comprising:
determining, by a controller of the surgical instrument, whether (i) a jaw assembly of the surgical instrument is in a closed state and (ii) a bipolar energy has not been applied by the surgical instrument;
determining, by the controller, an impedance of tissue captured within the jaw assembly;
comparing, by the controller, the determined impedance to a reference threshold impedance; and
instructing, by the controller, a user of the surgical instrument to reduce bending of the surgical instrument in response to a determination that the determined impedance of the tissue is greater than the reference threshold impedance.

20. The method of claim 19, wherein the reference threshold impedance is about 20 ohms.
